(19) United States
(12) Patent Application Publication
(10) Pub. No.: US 2026/0232462 A1
(43) Pub. Date: Aug. 13, 2026

(54) HYBRID URETHRAL STENT AND METHOD OF USE

(71) Applicant: Blue Halo BioMedical, LLC, Winter Park, FL (US)

(72) Inventors: Gaines Hammond, JR., Greenville, SC (US); Byron Hodge, JR., Lakeland, FL (US)

(21) Appl. No.: 19/426,871

(22) Filed: Dec. 19, 2025

Related U.S. Application Data
(63) Continuation-in-part of application No. 17/850,389, filed on Jun. 27, 2022.
Application No. 17/850,389 is a continuation-in-part of application No. 17/012,920, filed on Sep. 4, 2020, now abandoned.
(60) Provisional application No. 62/896,724, filed on Sep. 6, 2019.

(51) Int. Cl. A61F 2/82

(52) U.S. Cl. CPC A61F 2/82; A61F 2230/0004; A61F 2230/0091; A61F 2250/0059; A61F 2250/0067

(57) ABSTRACT

A hybrid urethral stent and method of inserting the hybrid urethral stent, wherein the hybrid urethral stent has a halo-shaped coiled portion extending away from a perpendicular stem portion through a swan neck portion. Eyelets on the halo coil portion and swan neck portion facilitate flow out of the bladder through the stent hybrid urethral stent vertical to the stent, to permit peri-tubular urine flow. The hybrid urethral stent is inserted into the body while straightened and thereafter return to its coiled shape when a stylet is removed.

### CROSS-REFERENCE TO RELATED APPLICATION

This application is a continuation-in-part of U.S. patent application Ser. No. 17/850,389, filed on Jun. 27, 2022 and entitled “COIL CATHETER, METHOD OF USE, AND METHOD OF MANUFACTURE” which claims priority to U.S. patent application Ser. No. 17/012,920, filed Sep. 4, 2020 and entitled “COIL CATHETER, METHOD OF USE, AND METHOD OF MANUFACTURE” which claims priority in U.S. Provisional Patent Application No. 62/896,724 filed Sep. 6, 2019 and entitled “HALO COIL CATHETER AND METHOD OF USE” all of which are incorporated herein by reference in their entirety.

### BACKGROUND OF THE INVENTION

### Field of the Invention

The present invention relates generally to a urinary collection and conveyance system comprised of a hybrid urethral stent with coil, swan neck and stem components, method of use and more specifically to a stent having a retention coil member for urine collection, method of placement and removal, positioning, validation of placement, and method of use. A hybrid urethral stent as used in this disclosure is comprised of a non-seating stent and, along with one or more eyelets within a stent body which permit urine flow bi-directionally through the eyelets and allows for peri-tubular and intra-tubular urine flow.

### Background

Urinary retention in males generally implies that urine is produced normally but is retained within the bladder due to primary detrusor dysfunction or an obstruction at or distal to the bladder neck. Prostatic obstruction causes an increased resistance for the passage of urine with subsequent increased pressure on the bladder musculature resulting in two successive phases; 1) compensation, where bladder emptying still takes place, and 2) decompensation, characterized by increasing amounts of residual urine and ultimately urine retention. Bladder outlet obstruction producing urinary retention may result from, including but not limiting, benign prostatic hyperplasia, prostate cancer, or any acute enlargement of the prostate including but not limited to acute prostatitis, post focal procedures on the prostate, radiation, cryotherapy or instrumentation. Further, the current standard of care to use a foley catheter with internal and external components including a balloon, tube and urine collection bag causes a host of potential problems that impact lifestyle and cause infection. Patients are susceptible to pressure induced ischemic necrosis of the bladder outlet caused by the balloon frequently resulting in encrustation. This current standard of care is designed to passively drain the bladder as compared to the present invention which allows for on-demand volitional voiding. Incomplete bladder emptying, urine retainment and stagnant urine, and internal and external components induce a host of problems to an already ill patient.

Urinary retention occurs primarily in males with benign prostatic hyperplasia being the most common cause in men over the age of 50 years. It has been estimated that one in four men in the United States will have been treated for symptomatic benign prostatic hyperplasia by the time they reach 80 years of age.

For patients with neurogenic bladders and females with urinary retention an embodiment of the hybrid urethral stent could be designed to facilitate urinary flow during a slight tug on the suture straightening the swan neck and pulling the stem distal to the external sphincter. Upon release of the suture the swan neck returns to its normal position and retracts the stem above the external sphincter recreating continence.

In an embodiment, a hybrid urethral stent capable of transforming a stent from a first, straight orientation to a second, coiled orientation, the hybrid urethral stent system comprising a hybrid urethral stent having a stent body including a proximal end and a distal end; the proximal end of the stent body comprising a single coil having a halo portion terminating into a swan neck element and a right-angle bend, wherein the swan neck element and the right-angle element are configured to form a stabilizing elbow, and wherein the proximal end terminates into a tapered tip; the distal end of the stent body comprising a stem and an extraction thread; the stabilizing elbow configured to allow for dynamic movement and further configured to provide a memory force to allow for snap back in proximity with a sphincter; the stabilizing elbow terminating into a straight stem culminating in the distal end of the stent body, the straight stem configured to stent a male prostatic or female urethra and to be placed above the external sphincter without penetrating the sphincter during use; and wherein the single coil being perpendicular to the straight portion; a single first eyelet located along a horizontal plane of the single coil; a single second eyelet located along the stabilizing elbow; wherein the stent body functions as a hybrid urethral stent while inserted in a body with the distal end located above the sphincter.

In yet another non-limiting embodiment, a method of inserting a hybrid urethral stent capable of transforming from a first, straight orientation to a second, coiled orientation, the method comprising providing the hybrid urethral stent having a stent body including a proximal end and a distal end; the proximal end of the stent body comprising a single coil terminating into a swan neck element and a right-angle bend, wherein the swan neck element and the right-angle element are configured to form a stabilizing elbow, and wherein the proximal end terminates into a tapered tip; the distal end of the stent body comprising an extraction thread; the stabilizing elbow configured to allow for dynamic movement and further configured to provide a memory force to allow for snap back in proximity with a sphincter; the stabilizing elbow terminating into a straight stem culminating in the distal end of the stent body, the straight stem configured to stent a male prostatic or female urethra and to be placed above the external sphincter at a base of a bladder without penetrating the sphincter during use; and wherein the single coil being perpendicular to the straight portion; a single first eyelet located along a horizontal plane of the single coil; a single second eyelet located along the stabilizing elbow; and wherein the stent body functions as a prosthetic urethral stent while inserted in a body with the distal end located above the sphincter; aligning, the hybrid urethral stent system stent in its first, straight orientation to facilitate insertion through a urethra; advancing, the hybrid urethral stent system into the urethra such that the proximal end of the stent body is positioned at the bladder base and the distal end of the stent body stents the male prostatic or female urethra and is placed above the external sphincter without penetrating the sphincter; transforming, the hybrid urethral stent system in its second, coiled orientation, wherein the single coil assumes a perpendicular configuration relative to the straight stem and the stabilizing elbow conforms to the anatomy near the external sphincter; and anchoring, the hybrid urethral stent system utilizing the memory force of the stabilizing elbow to maintain the hybrid urethral stent system in position within the body. In an embodiment, the stent body terminates proximal to the external sphincter.

Heretofore there has not been available a system or method for a hybrid urethral stent with the advantages and features of the present invention.

### BRIEF SUMMARY OF THE INVENTION

The invention involves facilitating drainage and, more specifically, but not by way of limitation, to facilitating urine drainage from the bladder through the urethra of a male or female patient experiencing either acute or chronic urinary retention. In an aspect, A hybrid urethral stent system capable of transforming from a first, straight orientation to a second, coiled orientation, the stent comprising a stent having a stent body including a proximal end and a distal end; the proximal end of the stent body comprising a single coil having a halo portion terminating into a swan neck element and a right-angle bend, wherein the swan neck element and the right-angle element are configured to form a stabilizing elbow, and wherein the proximal end terminates into a tapered tip; the distal end of the stent body comprising an extraction thread; the stabilizing elbow configured to allow for dynamic movement and further configured to provide a memory force to allow for snap back in proximity with a sphincter; the stabilizing elbow terminating into a straight stem culminating in the distal end of the stent body, the straight stem configured to stent a male prostatic or female urethra and to be placed above the external sphincter without penetrating the sphincter during use; and wherein the single coil being perpendicular to the straight portion; a single first eyelet located along a horizontal plane of the single coil; a single second eyelet located along the stabilizing elbow; wherein the stent body functions as a prosthetic or female urethral stent while inserted in a body with the distal end located above the sphincter. In yet another non-limiting aspect, A method of inserting a hybrid urethral stent capable of transforming from a first, straight orientation to a second, coiled orientation, the method comprising:
providing the hybrid urethral stent having a stent body including a proximal end and a distal end; the proximal end of the stent body comprising a single coil terminating into a swan neck element forming a right-angle bend, wherein the right-angle element is configured to form a stabilizing elbow, and wherein the proximal end terminates into a tapered tip; the distal end of the stent body comprising straight stem and an extraction thread; the stabilizing elbow configured to allow for dynamic movement and further configured to provide a memory force to allow for snap back in proximity with a sphincter; the stabilizing elbow terminating into a straight stem culminating in the distal end of the stent body, the straight stem configured to stent a male prostatic or female urethra and to be placed above the external sphincter at a base of a bladder without penetrating the sphincter during use; and wherein the single coil being perpendicular to the straight portion; a single first eyelet located along a horizontal plane of the single coil; a single second eyelet located along the stabilizing elbow; and wherein the stent body functions as a hybrid prosthetic or female urethral stent while inserted in a body with the distal end located above the sphincter; aligning, the hybrid urethral stent system stent in its first, straight orientation to facilitate insertion through a urethra; advancing, the hybrid urethral stent system into the urethra such that the proximal end of the stent body is positioned at the bladder base and the distal end of the stent body stents the urethra and is placed above the external sphincter without penetrating the sphincter; transforming, the hybrid urethral stent system in its second, coiled orientation, wherein the single coil assumes a perpendicular configuration relative to the straight stem and the stabilizing elbow conforms to the anatomy near the bladder neck; and anchoring, the hybrid urethral stent system utilizing the memory force of the stabilizing elbow to maintain the hybrid urethral stent system in position within the body.

The directional terms proximal and distal require a point of reference. In this application, the point of reference in determining direction is from the perspective of the patient. Therefore, the term proximal will always refer to the point that is closest to the center or trunk of the human body, whereas distal will always refer to the point that is farthest from the center or trunk of the human body relative to the other point.

The hybrid urethral stent is primarily comprised of a proximal coil, swan neck and distal stem component. Generally, the proximal collection member for the hybrid urethral stent is coil-shaped, the mid-section retention member for the hybrid urethral stent is a swan neck, and the distal straight section of the hybrid urethral stent is a stem. The hybrid urethral stent has a straight non coiled iteration for placement. Once placed into the bladder a stylet is removed which allows for the coiled default state to be achieved. The pusher segment is connected to the stylet during placement and pusher abuts the distal end of the uncoiled hybrid urethral stent. The pusher may aid in pushing the hybrid urethral stent into position. In an embodiment, the diameter of the pusher may be smaller than the external but not internal diameter of the hybrid urethral stent.

In general, and in one aspect, the invention relates to a stent system, which embodies the ability to control a coil tip hybrid urethral stent within the bladder or body cavity by an attached thread traversing through the urethra to the exterior. The hybrid urethral stent comprises a proximal coil member, a swan neck retaining member and distal stem straight member. The hybrid urethral stent tube has a lumen extending throughout the hybrid urethral stent from the proximal coil's tapered end to the end of distal straight non-tapered stem to allow fluid drainage through the hybrid urethral stent as well as around the straight stem segment. “Eyelets” as used in this disclosure, are one or more holes including perforations, holes, orifices are placed in the hybrid urethral stent at, but not limited to, along the coil to the tip, the swan neck section and the straight stem. A tapered opening in the proximal hybrid urethral stent allows the use of a guide wire to facilitate delivery of the hybrid urethral stent into the bladder due to urethral or prostate anatomical challenges.

The hybrid urethral stent is sized for placement substantially within the bladder and bladder neck, prostate urethra, with the distal terminating end located proximal to the external urethral sphincter to allow normal operation of the external sphincter. The coil and swan neck retaining members extend from the proximal end portion of the hybrid urethral stent to the bladder outlet where the stem continues into the male prostatic or female urethra. The coil and swan neck retaining members are straightened into a first state to allow passage of the stent into the urethra and bladder, and, upon removal of the stylet, the coil retaining member is coiled into a second state when located in a bladder to hold member in place substantially within the urethra by removing a straightening stylet. The right angle and swan neck configuration allows for retaining of the hybrid urethral stent as well as a tolerance to traction prior to removal. The pusher segment abuts to the distal tubular segment to hold the hybrid urethral stent in position as the straightening stylet is removed.

Embodiments of this aspect can include the following features. The hybrid urethral stent system is comprised of a hybrid urethral stent, a monofilament suture, and a pusher segment. The hybrid urethral stent is comprised of a hybrid urethral stent body including a distal end and a proximal end, the proximal end comprising a single coil having a halo portion terminating into a swan neck element and a right-angle bend and the distal end comprising a stem connected to an extraction suture to its distal end, and a proximal coil retaining member. An “anti-migration suture” as used in this disclosure, is attached to the distal stem to anchor the hybrid urethral stent within the urethra and aid in positioning, stabilizing and removal of the hybrid urethral stent. The Swan Neck portion of the hybrid urethral stent is the transition from the coil as it angles proximally then curves distally into a horizontal straight stem portion of the hybrid urethral stent. The suture is a non-absorbable monofilament.

The length of the stem, swan neck and coil can be changed to facilitate a variety of anatomical and gender specific challenges. Multiple coils and a short tubular segment is appropriate for females with short urethras which would allow for stenting from inside the bladder outward and controlled externally with the anti-migration control suture. In patients with a dysfunctional sphincter, the hybrid urethral stent requires bridging the sphincter to empty the bladder then snap back proximal to the sphincter to allow for continence. The default state for spinal cord and female patients similar to above would be proximal to the external urinary sphincter and allow dynamic bladder emptying by pulling on the suture to bridge the stem across the sphincter. Upon bladder drainage in these types of patients, the distal end of the stem, upon release of the suture, will revert back to a position proximal to the sphincter which allows for continence.

The coil and swan neck replace the balloon as a retaining member. The coil consists of a tapered tip with guide wire channel and eyelets placed at various locations but not limited to the coil segment. The eyelets may be placed at various locations on the coil and the swan neck to minimize direct contact with the mucosa. At the juncture of the coil and stem is a “swan neck” portion which extends from the horizontal plane of the coil hybrid urethral stent which is a right angle to the tubular stem member. The “Swan Neck” portion allows for some allowance for traction on the hybrid urethral stent prior to the straightening of the horizontal coil member. While the removal of an inflated balloon type catheter or stent results in bladder outlet and urethral mucosal injury, the hybrid urethral stent is designed intentionally to be removed by applying traction on the suture. Therefore, planned or inadvertent removal does not result in mucosal injury since the diameter of the hybrid urethral stent at removal is the same as upon insertion.

The coil and swan retaining members of the hybrid urethral stent can be a tube constructed with form-shaped memory. The coil retaining member also can be rounded at the ends of the tubing to provide user comfort during insertion into the patient's urethra. A guide wire channel allows for a guide wire to be utilized with difficult placements due to false passages in the urethra or other anatomical challenges. In an embodiment, the hybrid urethral stent may be inserted with the use of a cystoscope. A “cystoscope” as used in this disclosure, is any medical instrument that allows doctors to look inside a urinary tract, such as by examining a bladder and urethra.

The process for creating the swan neck vertical dynamic portion of the hybrid urethral stent with a horizontal coil is a unique process encompassing features defined in the Utility patent identified as Manufacturing Process for integration of retaining member with vertical component coupled with the horizontal coil. The Vectors of pull impact the hybrid urethral stent in two separate phases of uncoiling. They are coupled with a resistance to pull out which is initially absorbed by the vertical swan neck and the single coil.

Prior to and during insertion of the hybrid urethral stent into the patient's urethra, the coil and swan neck retaining members are in a straightened first state. Once in the patient's bladder, removal of the guide wire and stylet allow the retaining members to return to substantially the second coil and swan neck state and thereby act as anchors to keep the stem of the stent substantially within the male prostatic or female urethra.

In one embodiment, the stem can include one or more eyelets to allow fluid to drain from the stem into the urethra creating additional peri-tubular drainage to flush the prostate and urethra. The lumen of the body member and length of the tube can be designed to be equivalent to a variety of tubular dimensions.

In one embodiment, the suture should be long enough to extend from the distal portion of the stem to the outside of the patient's body. The suture may serve as an anti-migration component to prevent migration of the hybrid urethral stent into the urethra and bladder. The suture may also aid in tethering, insertion, dynamic positioning and removal. The hybrid urethral stent can be removed easily and without injury from the patient's body by pulling downward on the suture. The end of the suture may be connected to an anti-migration structure (e.g., snap cap, ball, ring, coil) that extends out of the body entirely. The purpose of the snap cap is useful for hybrid urethral stent positioning, confirmation of positioning, and anti-migration. It may also be used to facilitate location of the suture's external end and aid in hybrid urethral stent removal by simply pulling on the located suture. Gentle traction on the snap cap is sufficient. In patients with diminished manual dexterity, a magnet can be placed on the snap cap in order to facilitate location and retrieval. This allows for engaging the snap cap with gentle traction to traverse the sphincter in male and female patients with external sphincter resistance.

The hybrid urethral stent may utilize additional materials which would add qualities such as, but not limited to, lubrication, hydrophilic coating, pharmaceuticals and radiographic enhancing material. The material for the hybrid urethral stent includes, but is not limited to, Carbothane. The internal and external diameters (ID and OD) may be of various sizes, stiffness and materials. The pusher, stylet and suture material are not limited in size or characteristics. The suture may also be comprised of monofilament nylon or other equivalent materials.

In another embodiment, the invention relates to a hybrid urethral system for draining fluid, purulent or otherwise, from a patient's body cavity including but not limited to bladder, stomach, kidney, colon, ileal loop, colostomy, and abdominal peritoneal cavity.

In general, in still another aspect, the invention allows for the manipulation of a variety of hybrid urethral stents within the body cavity to be controlled externally with a tethering suture. This allows for episodic movement from a passive a dynamic state of the hybrid urethral stent, which changes the fluid dynamics to favor removing a collection of fluid from the body or a cavity, for example, a urinary diversion.

Another embodiment includes the capability of the hybrid urethral stent being imbedded with various pharmaceuticals which allows for a unique drug delivery into body cavities such as, but not limited to, the prostate, kidney, bladder, stomach, colon, ileal loop, colostomy or abdominal cavity. For example, medications such as chemotherapy and antibiotics and other pharmaceuticals may be delivered directly into a body cavity. The tethered control also allows for manipulation of the hybrid urethral stent in the cavity. The hybrid urethral stent may be configured with monitoring devices allowing for the wireless transmission of images or data.

In another embodiment, pharmaceuticals may be applied to or incorporated into the hybrid urethral stent using a combination of physical, chemical, and biological methods, depending on the drug's chemistry and the delivery goals including, but not limited to, local versus systemic, fast versus sustained release, or targeted versus passive. Applications and delivery methods using the hybrid urethral stent may include, but are not limited to, physical incorporation (entrapment), surface adsorption, chemical conjugation (covalent attachment), encapsulation in vesicular or biological carriers, affinity-based binding, impregnation or coating of solid devices and advanced triggered release systems.

In another embodiment, custom tumor mapping allows spatially and biologically applying drugs to specific areas of the hybrid urethral stent segments to target drug release to specific tumor locations. Drug loads or drugs can also vary on different segments of the hybrid urethral stent to coordinate drug delivery to asymmetric or focal tumors sparing healthy tissue. Anatomical mapping and drug delivery matching strategies optimize safety and effectiveness by tumor-specific targeting methodologies which spare healthy tissues and maximize drug utility.

The hybrid urethral stent facilitates volitional voiding by allowing the bladder to fill, contract with synchronous sphincter relaxation and empty with minimal prostate urethral resistance. This allows for evaluating the functional capacity of both the bladder and urinary sphincter. In patients with chronic over distension due to prostate obstruction, the hybrid urethral stent may act as a bladder rehabilitation device by dilating the prostate fossa, and promoting engagement of the detrusor muscle, and internal and external sphincter muscle functionality. With acute urinary or chronic retention, dilating the prostate fossa during insertion of the hybrid urethral stent may alleviate obstruction post-removal and obviate the need for a variety of interventional procedures. More efficient voiding, with reduced residual urine in the bladder, coupled with a competent urinary sphincter and elimination of the need for an external-collection device results in a collage of clinical improvements. In female patients with chronic urinary retention, this hybrid urethral stent may similarly improve bladder function. The hybrid urethral stent aids in fostering bladder function preservation so that once the hybrid urethral stent is removed a patient may be voiding on his or her own. Throughout use of the hybrid urethral stent the bladder continues to function in conjunction with internal and external sphincter functionality to preserve muscle functionality.

With other coils the uncoiling began with minimal tension on the distal tubular portion. The current innovation protects the uncoiling of the horizontal component from minimal tension. The “swan neck” portion allows for lengthening of the vertical tubular component and with release of the tension will “snap back” to its original position due to the horizontal stabilizing effect. The distance for the extension and snap back allows for a variety of medical applications with innate resistance to inappropriate migration which is common in “pig tail curl” or “J” shaped tips. In an embodiment, the hybrid urethral stent may include a double “J” embodiment wherein the proximal end and/or the distal end of the stent body may contain a “J” shaped tip. In such an embodiment, this may aid in preventing migration of the hybrid urethral stent within the human body. The application defines the unique manufacturing process to construct the vertical-swan neck-horizontal shaped hybrid urethral stent.

### BRIEF DESCRIPTION OF THE DRAWINGS

The drawings constitute a part of this specification and include exemplary embodiments of the present invention illustrating various objects and features thereof.

FIG. 1 is a diagrammatic representation of a preferred embodiment of the present inventions shown in a typical environment.

FIG. 2 is a front elevational view of a preferred embodiment of the present invention shown in a straightened orientation.

FIG. 3 is a top plan view thereof.

FIG. 4 is a bottom plan view thereof.

FIG. 5 is a top plan view of a preferred embodiment of the present invention in a coiled orientation.

FIG. 6 is a front elevational view thereof.

FIG. 7 is a three-dimensional isometric view thereof.

FIG. 8 is a front elevational view of a preferred embodiment of the present invention in combination with a typical pusher device.

FIG. 8A is a detailed view of a portion thereof taken about the circle 8A in FIG. 8.

FIG. 9 is a diagrammatic representation thereof shown in a typical environment with an external container.

FIG. 9A is a detailed view of a portion thereof taken about the circle 9A in FIG. 9.

FIG. 10 is a diagrammatic representation thereof shown without an external container.

FIG. 10A. is a detailed view of a portion thereof taken about the circle 10A in FIG. 10.

FIG. 10B is a detailed view of a portion thereof taken about the circle 10B in FIG. 10.

FIG. 11 is a three-dimensional view showing the preferred embodiment of the present invention in combination with a pusher.

FIG. 11A is a detailed view of a portion thereof taken about the circle 11A in FIG. 11.

FIG. 12 is a three-dimensional view showing a manufacturing step for manufacturing a preferred embodiment of the present invention.

FIG. 13 is a three-dimensional isometric view showing a second manufacturing step thereof.

FIG. 14 is a three-dimensional isometric view showing a third manufacturing step thereof.

FIG. 15 is a three-dimensional isometric view showing a fourth manufacturing step thereof.

FIG. 16 is a three-dimensional isometric view showing a fifth manufacturing step thereof.

FIG. 17 is a three-dimensional isometric view showing a sixth manufacturing step thereof.

FIG. 18 is a three-dimensional isometric view showing a seventh manufacturing step thereof.

FIG. 19 is a flow chart diagramming the method of using a preferred embodiment of the present invention.

FIG. 20 is a flow chart diagramming the method of manufacturing a preferred embodiment of the present invention.

FIG. 21 is a flow chart diagramming a method of inserting a halo-style stent.

### DETAILED DESCRIPTION OF THE PREFERRED EMBODIMENTS

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

The directional terms proximal and distal require a point of reference. In this application, the point of reference in determining direction is from the perspective of the patient. Therefore, the term proximal will always refer to the point that is closest to the center or trunk of the human body, whereas distal will always refer to the point that is farthest from the center or trunk of the human body relative to the other point.

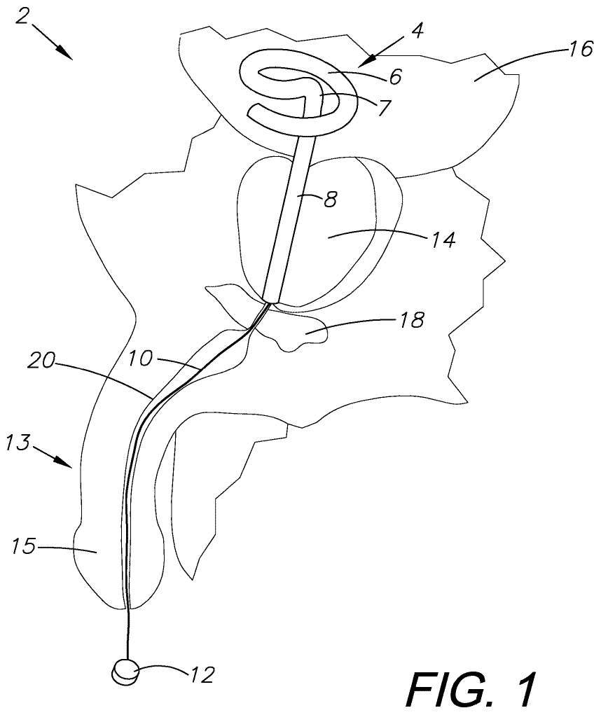

As shown in a typical environment in FIG. 1, the present invention is a stent system 2 which features a coiled stent 4 having a halo portion 6 connected to a stem portion 8 via a swan neck bend 7 such that the halo portion is along a plane perpendicular to the direction of the stem portion. A “stent system” as used in this disclosure is any system that is inserted into the body of a human and/or animal to allow the movement of fluids and/or access to internal structures for various medical purposes such as drug delivery. A stent system may include any system previously referred to as a catheter. The halo portion 6 is located within the bladder 16 once properly placed and serves to provide optimal flow out of the stent 4 through the stem 8. In an embodiment, the coiled stent 4 may be placed within the bladder base. FIG. 1 shows a thread 10 connected to a snap cap 12 located outside of the body to prevent the thread 10 from being drawn up into the body and instead would be stopped at the glans 15 of the penis 13 (as shown). This would function similarly in a female patient. The thread would be formed from a monofilament suture-type material in a preferred embodiment. A “monofilament suture” as used in this disclosure, is a surgical thread made from a single smooth strand of material, designed to minimize tissue trauma and reduce infection risk. The monofilament suture may be made from one continuous strand, giving it a smooth surface. The smooth surface may allow the suture to glide easily through tissue, causing less damage during insertion. The smooth surface may prevent the aggregation of bacteria as compared to a braided suture, making the suture an ideal material to be placed in the urogenital area where there can be large quantities of bacteria nearby. The monofilament suture may be composed of one or more materials including but not limited to nylon, polypropylene, polydioxanone, and/or poliglecaprone. In an embodiment, the monofilament suture may be absorbable by the body. In an embodiment, the monofilament suture may be non-absorbable and may remain in the body unless removed. The snap cap 12 may include a magnet or be made of magnetic material. The stem portion 8 is configured to stent the male prostatic or female urethra and is adapted to permit a peri-tubular urine flow. The stem portion 8 is placed above the external sphincter to preserve volitional voiding. “Volitional voiding” as used in this disclosure, is any conscious and intentional act of urinating, where an individual initiates and controls bladder emptying through voluntary muscle coordination. Volitional voiding may include the ability to sense bladder fullness, decide when to urinate, and activate pelvic floor and bladder muscles to release urine.

The preservation of volitional voiding during insertion of the hybrid urethral stent may preserve and continue to engage a detrusor muscle contraction functionality and the internal and external sphincter functionality. This may provide several key benefits, including aiding in more fully emptying the bladder; flushing microbial biofilm and contaminants from the bladder and urethra; and preventing disuse atrophy and potentially contributing to temporary or permanent post-removal volitional voiding functionality. The stem portion 8 terminates in the male prostatic or female urethra proximal to the external voluntary sphincter thereby preserving bladder mechanics including external voluntary sphincter, internal involuntary sphincter, detrusor muscle functionality, and urge to void sensation. “Peri-tubular urine flow” as used in this disclosure, is the flow and movement of substances between the kidney's peritubular capillaries and the renal tubules, permitting urine formation and ensuring adequate reabsorption and secretion processes. Peri-tubular urine flow includes the absence of a non-obstructing anchor such as a balloon at the bladder outlet such as for example as seen with a foley catheter. This further allows for an absence of stagnant urine pool in the bladder and an absence of external catheter components ultimately allowing for the creation of a normal urinary state which contributes to the displacement of pathogenic bacteria by flushing the urinary tract system and preserving volitional voiding. Additionally, this aids in preventing bladder necrosis such as when parts of the bladder may lose their blood supply due to ischemic pressure necrosis or are directly damaged ultimately leading to cell death. In addition, the hybrid urethral stent is made of less erosive materials, and it is smaller and more flexible. The standard of care in practice to use a foley catheter containing a balloon may cause pressure necrosis due to ischemia which opens a patient up to bacteria exposure and infection. Peri-tubular urine flow and improved bladder emptying may aid in reducing the incidence of urinary tract infections (UTIs) for patients who use the hybrid urethral stent. For example, in 80-90 various clinical trials, patients started with approximately 500 cc of pre-void residual. Once the hybrid urethral stent was inserted, the average post void residual was 20 cc or less and is defined as complete bladder evacuation. This is drastically more effective than those catheterized with a balloon obstructing foley catheter. The balloon obstructing foley catheter has not been shown to lower the post void residual below the tip of the catheter which terminates above the bladder-outlet-obstructing balloon. It only eliminates the overflow and leaves 100 cc or greater of stagnant urine in the bladder. Peri-tubular urine flow ensures an adequate exchange of substances between the blood in the capillaries and the tubular fluid which becomes urine. Peri-tubular urine flow ensures there is a balance maintained of fluid and electrolytes, regulation of blood pH, elimination of toxins and drugs, and concentrates urine by reclaiming water. Peri-tubular urine flow may allow for urine follow through from the base of the bladder across the internal sphincter and through the prostate and urethra by any method. Peri-tubular urine flow may displace pathogenic bacteria from the urinary tract. This is further enhanced by urine flowing through the urethra during voiding several times daily as in the natural state to flush the urethral mucosa and external stent tubing of microbial contaminants and biofilm accumulation. Peri-tubular urine flow may aid in moisturizing the urinary tract and protecting the mucosal barrier within the urinary tract. The swan neck bend 7 serves as an anchoring feature to position the stent system 2 whereby the coiled stent 4 is positioned at the bladder base. An “anchoring feature” as used in this disclosure, is a structural or mechanical component designed to secure the stent system 2 within the body, thus preventing unwanted movement and ensuring accurate function. Anchoring feature may ensure stability to keep the stent system 2 at the intended site within the body to maintain the coiled stent 4 at the bladder base. Anchoring feature may function as a safety mechanism to reduce the risk of migration dislodgement, or tissue damage. Anchoring feature may include pigtails, figure of eight, and/or any other suitable anchoring feature. A pigtail may include a coil and/or spiral shaped tip designed to secure the stent system 2 within the body. The pigtail may resemble a curled tail or loop. The pigtail may anchor and stabilize the stent within the bladder base and provide a cushion against tissue, reducing irritation or injury. A figure of eight may include a structural design resembling the shape of the number 8, used to stabilize and secure the stent system 2 within the body, by anchoring securely to soft tissue, preventing movement of the stent system 2, and distributing tension or load evenly across contact points. Positioning the coiled stent 4 at the bladder base creates a non-obstructive placement at the bladder outlet and allows urine to flow through the stent and peri-tubularly around it through the male prostatic or female urethra providing novel and unique dual flow pathways. Anchoring feature may include various other anchor designs including but not limited to an umbrella, octopus legs, petals, and the like.

The stent 4 stem 8 passes through the prostate gland 14 and the end of the stem is located in proximity with the external sphincter 18. This system facilitates flow from the bladder 16 through the stent 4 located at least at the swan neck section 7 and at the proximal coil tip of the coil portion 6, and out through the urethra 20. It is important that the stem 8 functions as a short straight arm that un-obstructs the male prostatic or female urethra and sits above the urinary sphincter 18. It does not retain the urinary sphincter in an open orientation ever. This allows for volitional voiding of the bladder by the patient, facilitated by the internal stent 4, without incontinence.

As shown in FIG. 1, when the stent 4 is inserted into the bladder, the coil portion 6 coils, forming the swan neck portion which terminates into a right-angle bend which together form a stabilizing elbow which ensures the stent remains properly in place within the bladder for optimal drainage through the stent. The stem 8 may include a eyelet. A “eyelet” as used in this disclosure, is a hole made through the surface of the stem 8. The eyelet may allow urine that enters the stent 4 from the bladder to escape into the urethra and help flush the urethra.

This may ensure that the prostate and urethra are flushed with urine approximately eight times per day, mimicking the body's natural state whereby there is a natural urinary tract cleansing system and reduces the risk of microbial infections. In an embodiment, the eyelet may be contained within the upper portion of the stem 8, closer to the bladder 16. The stent system 2 may be configured to accommodate a urine flow ranging from approximately 5 cubic centimeters per second to 30 cubic centimeters per second. The stent system 2 may be configured to accommodate a urethral length from approximately 2 centimeters to 30 centimeters. The stent system 2 may be comprised of a material selected from the group consisting of carbothane, silicone, latex, coated latex, hydrogel polyurethane, polyvinyl chloride (PVC), teflon, nylon, and an antimicrobial coating. An antimicrobial coating may include but is not limited to silver-based coatings, antibiotic loaded polymer coatings such as rifampicin, minocycline, and gentamicin, heparin coatings, nitric oxide releasing coatings, and bio-inspired polymer coatings such as hydrophilic polymers, zwitterionic coatings, and chitosan-based layers. In an embodiment, the entire stent 4 may be composed of 16F carbothane tubing. Carbothane is a medical grade thermoplastic polyurethane, a class of polymers known for superior performance and integrity in biomedical applications. Carbothane may have a higher resistance to encrustation including the formation of mineral deposits and biofilm development as compared to other materials typically used for stents. Carothane may also be less abrasive to the urethral mucosa than other materials typically used in other stent systems and may resist degradation from bodily fluids, oxygen, and enzymes to support long-term performance in the body. Carbothane may be well-tolerated by human tissue, making it suitable for long term use whereby the stent system 2 may be kept within the body for a duration of 30 days or more with additional extensions of time. In an embodiment, the stent system 2 may be replaced after a prescribed period of time. Carbothane may provide for superior tensile strength and elongation, allowing the stent system 2 to withstand stress and movement without tearing.

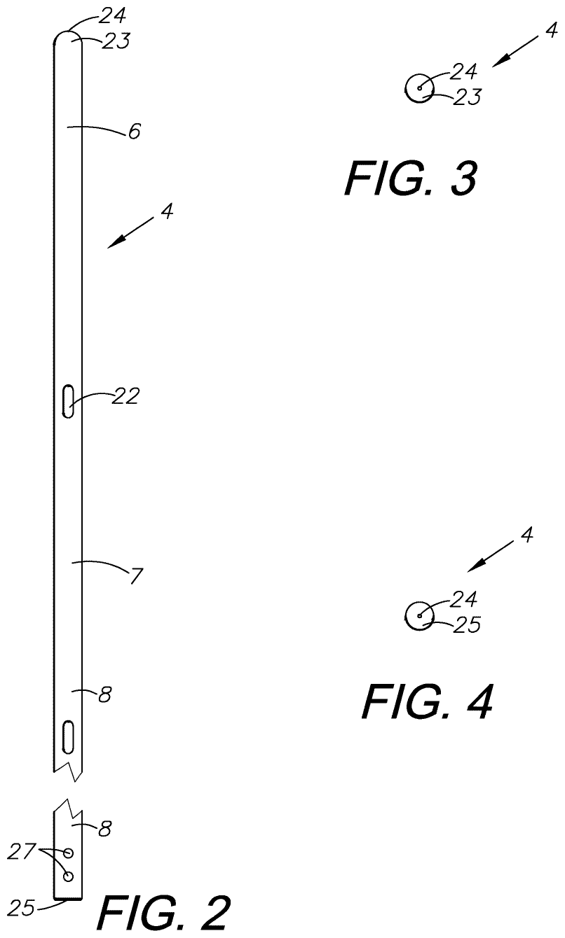

FIGS. 2-4 show how the stent 4 would be inserted into the body in a straight orientation for easy placement. Referring first to FIG. 2, a guidewire hole 24 is located at the tip 23 of the coil portion 6 and at the base 25 of the stem portion 8 for use with a pusher 26 and stylet 30 as is typical. This system facilitates flow from the bladder 16 through the stent 4 via eyelets 22, located at least at the swan neck section 7 and at the proximal coil tip 23 of the coil portion 6, and out through the urethra 20. As shown in FIG. 2, the tip 23 is tapered for easy insertion. Referring next to FIG. 3, a top view of guidewire hole 24 and the tip 23 is illustrated. Referring now to FIG. 4, a bottom view of guidewire hole 24 and the tip 23 is illustrated.

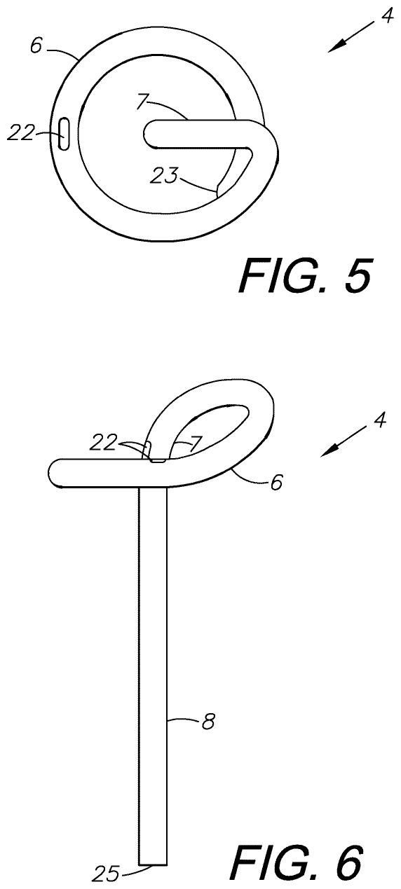

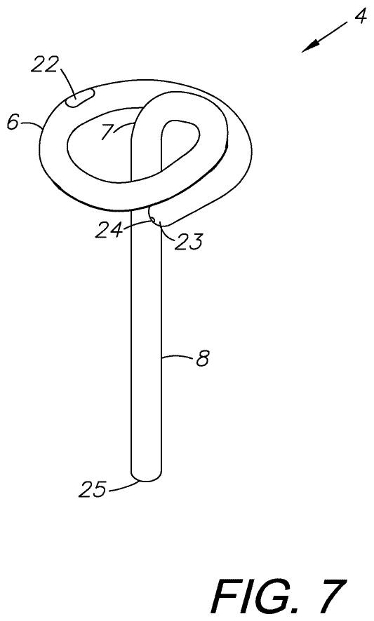

Referring now to FIG. 5, a top plan view of the stent 4 in the second coiled orientation showing the halo portion 6 and swan neck 7 connecting to the stem portion 8. The stent 4 forms into a coiled stent with a halo portion 6 and swan neck 7 connecting to the stem portion 8 once inserted into its proper environment in the bladder 16. The stent 4 defaults to this form due to a manufacturing process discussed below. Once the guidewire of the stylet 30 and pusher 26 elements is removed or at least withdrawn slightly, the stent 4 will automatically coil into the form shown. The second coiled orientation facilitates a complete bladder evacuation or near normal post void residuals by enabling drainage at the bladder base. Referring now to FIG. 6, a front elevational view of the halo portion 6 and swan neck 7 connecting to the stem 8 is illustrated. One or more eyelets 22 allow urine to flow through enabling drainage at the bladder base. Referring now to FIG. 7, a three-dimensional isometric view showing the halo portion 6 and swan neck 7 connecting to the stem portion 8 is illustrated. Tip 23 is illustrated along with guidewire hold 24. Eye 22 allows for urine flow to flow through and enables drainage at the bladder base. Base 25 of the stent 4 is illustrated.

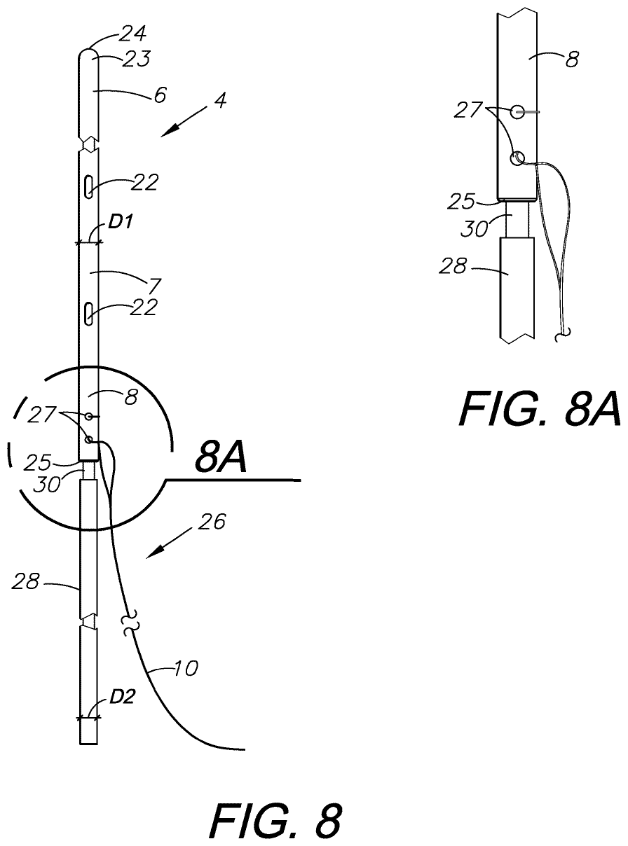

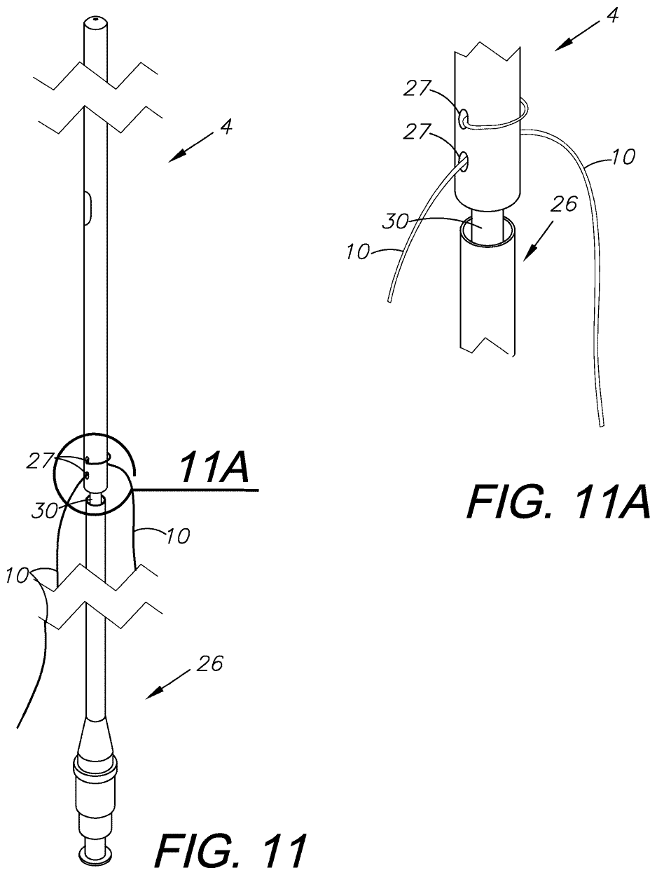

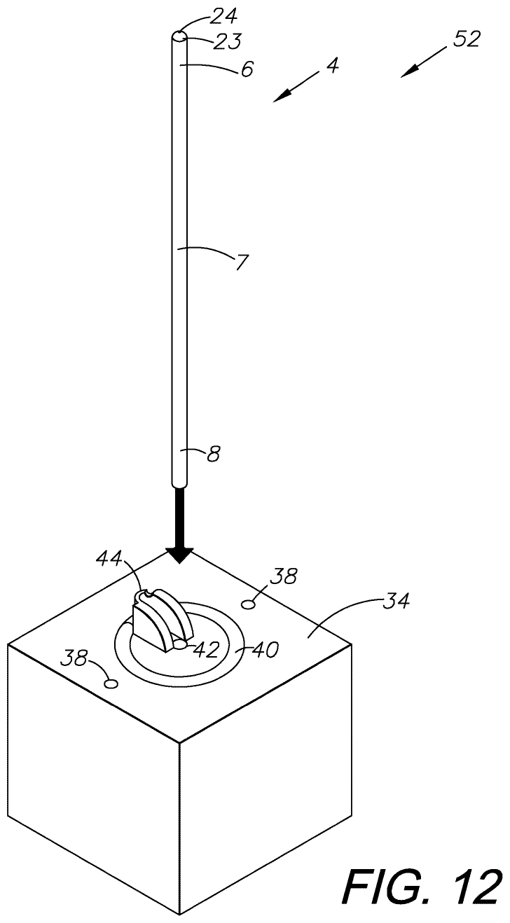

FIG. 8 is a front elevational view of the stent 4 in combination with a pusher device 26. The stent 4 in its straight orientation in combination with a pusher 26 having an outer tube 28 and a stylet 30. The thread 10 and snap cap 12 extend through the pusher 26 and connect to the end of the stent 4 near its base 25, shown in more detail in FIG. 11. These figures also show how the pusher 26 has a smaller diameter than that of the stent 4, further easing insertion of the stent. The straight orientation of the stent 4 is employed during insertion of the stent 4 into a subject. The stylet 30 serves as a guidewire that aids in transforming the stent 4 from a first, straight orientation to a second, coiled orientation. A “guidewire” as used in this disclosure, helps navigate through the body's vessels and/or ducts to create a pathway for the placement of a larger instrument such as the stent 4 and ensure proper placement within the urinary tract. In an embodiment, the guidewire may be used in self-stenting, such as when a patient may need to insert the stent 4 into his or her own bladder through the urethra to drain urine. The guidewire may accommodate a round tip to prevent the penetration of tissue during difficult insertions. Draining the bladder includes fully emptying of the bladder with drainage capability at the base of the bladder with peri-tubular urethral flow or any flow through the prostate and urethra with any method. In an embodiment, the stem 8 may terminate just above the external sphincter. In an embodiment, the stem 8 may terminate just distal to the external sphincter. In addition, this aids in maintaining the integrity of the urinary tract to prevent infection by eliminating stagnant urine with a lower drainage system. This also eliminates the need for an external stent tube resulting in a natural flushing of harmful bacteria through the urinary tract. This may aid in creating the ability to flush the prostate and urethra with urine eight times a day, mimicking a natural state of self-cleansing of the urinary tract system. Self-stenting may be utilized in patients who have urinary retention, neurogenic bladder, post-surgical complications, bladder dysfunction from diabetes and other chronic conditions and the like. The detailed view of FIG. 8A shows how the guidewire is retained through receivers 27 in the stent stem 8.

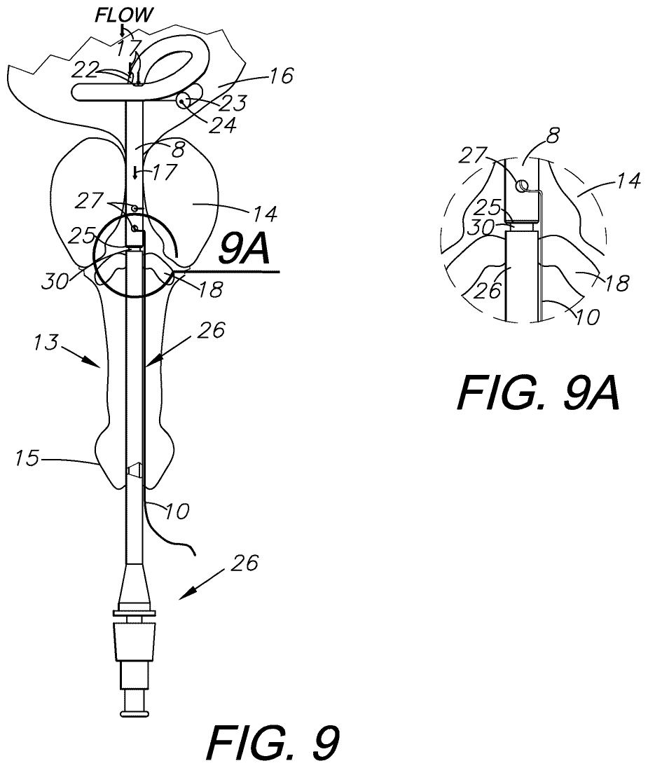

Referring now to FIG. 9, FIG. 9 shows the stent 4 and pusher 26 in the typical environment shown in FIG. 1 where the stent is fully inserted and coiled in position. Flow 17 is indicated via the arrows through the stent 4 by way of eyelets 22, vertically downward rather than perpendicular to the stent as is the case with prior art stents. The detailed view of FIG. 9A shows how the thread 10 is retained through receivers 27 in the stent stem portion 8 to secure the thread to the stent 4 for removal. The thread is shown external to the pusher 26, which abuts the base 25 of the stent 4 with the stylet 30 making contact with the base of the stem portion 8 thereof.

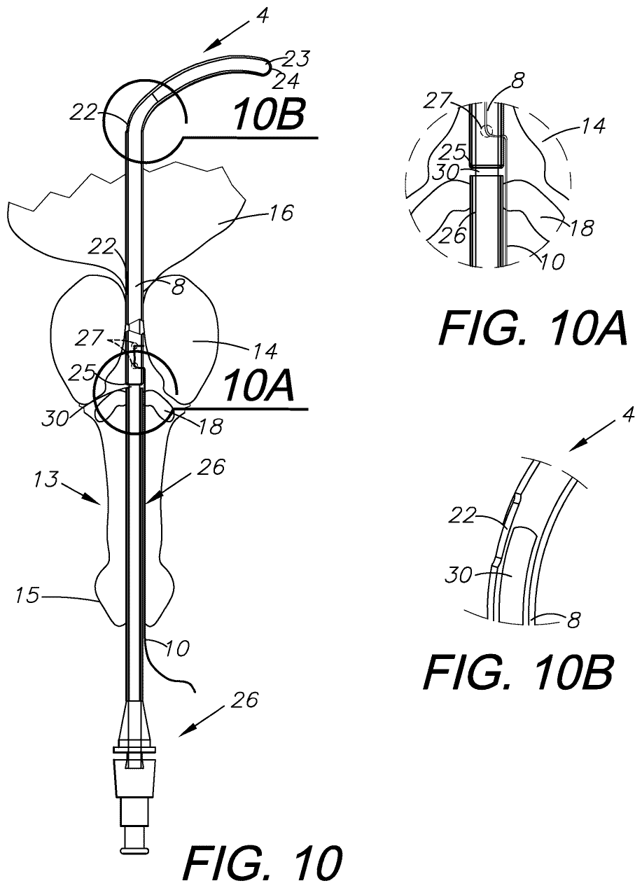

Referring now to FIG. 10, FIG. 10 shows the insertion of the stent 4 using the pusher 26 in a sectional view so as to better show the internal components thereof. FIGS. 10A and 10B show additional detail about their respective circles in FIG. 10, such as FIG. 10B showing how the stylet 30 of the pusher is a functional guidewire that can be placed up and into the stent itself to help position it within the bladder. The stent 4 is straightened by the internally placed stylet 30 with guidewire. The stent as shown is placed over the guidewire for safe insertion into the bladder, after which the guidewire and stylet are removed leaving only the coiled and unencumbered stent 4 in the bladder as shown in FIG. 1.

FIG. 11 and FIG. 11A show in more detail the extra-luminal suture thread 10 which does not obstruct the lumen of the stent 4. Like the positioning of the stem 8 about the sphincter 18, this is intended to prevent incontinence and is used to safely remove the stent at a later date. No sheath is required for such procedure.

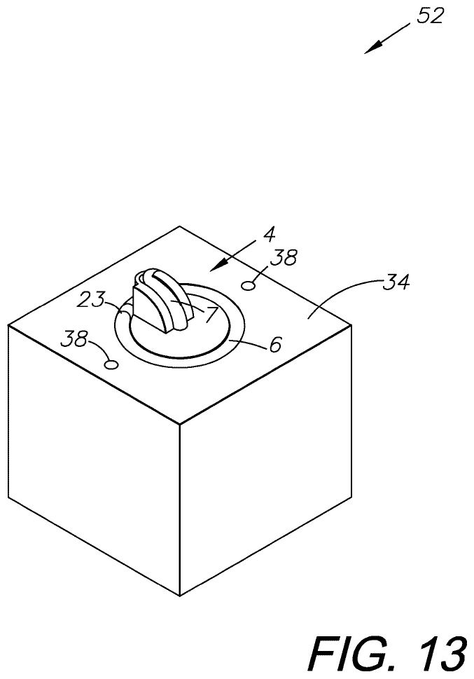

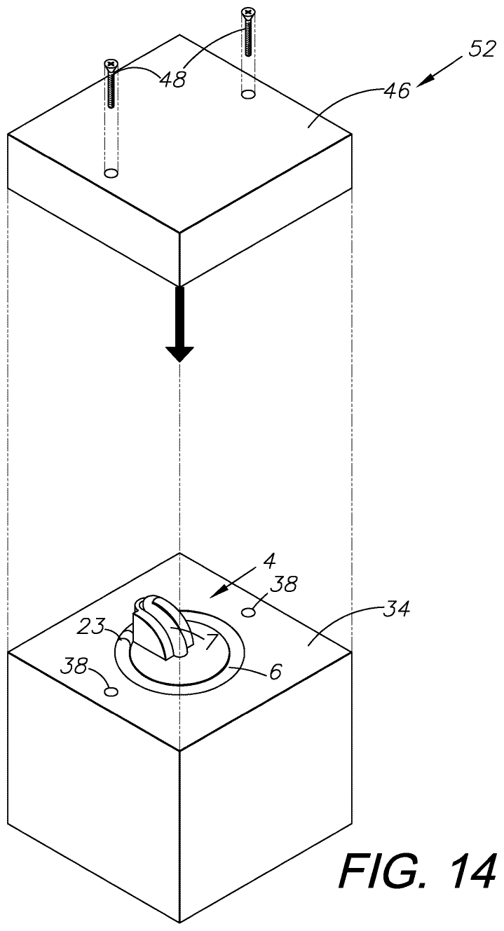

FIGS. 12-18 show a manufacturing system 52 for manufacturing the coil stent 4. As shown, the coil stent 4 is formed from an originally straight stent tube by placing it into a mold base 34. A flexible yet solid tubing support may be inserted into the stent tubing prior to molding to prevent kinks during the forming process. The mold base 34 has receiver slots 38 for screws 48 to receive the mold cap 46 as shown in FIG. 14. The mold base 34 also has a forming block with a swan neck form 44 and halo form 40. The stem 8 is inserted into the stem receiver 42 and the swan neck 7 portion is placed into the swan neck form 44, and the halo portion 6 is curled around in the halo form 40. The mold cap 46 is then secured to the mold base 34 via the screws 48.

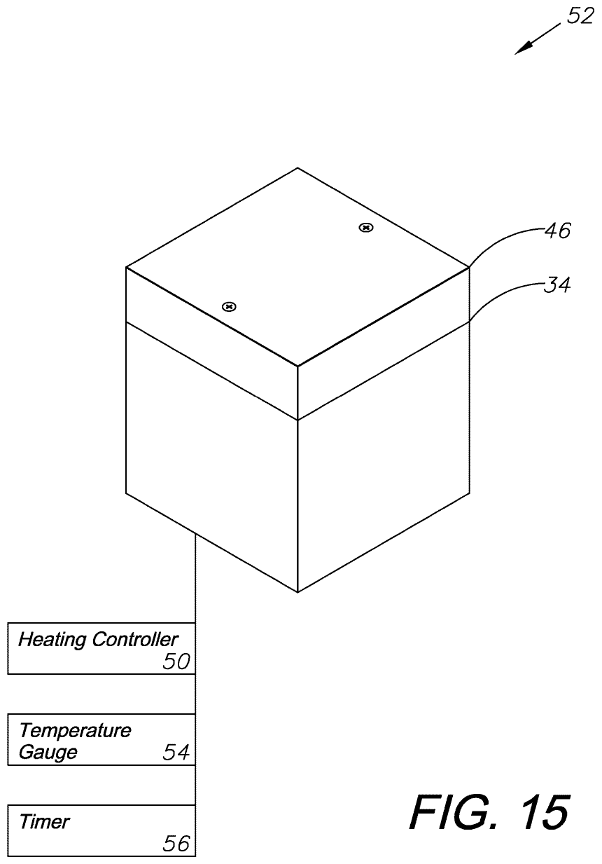

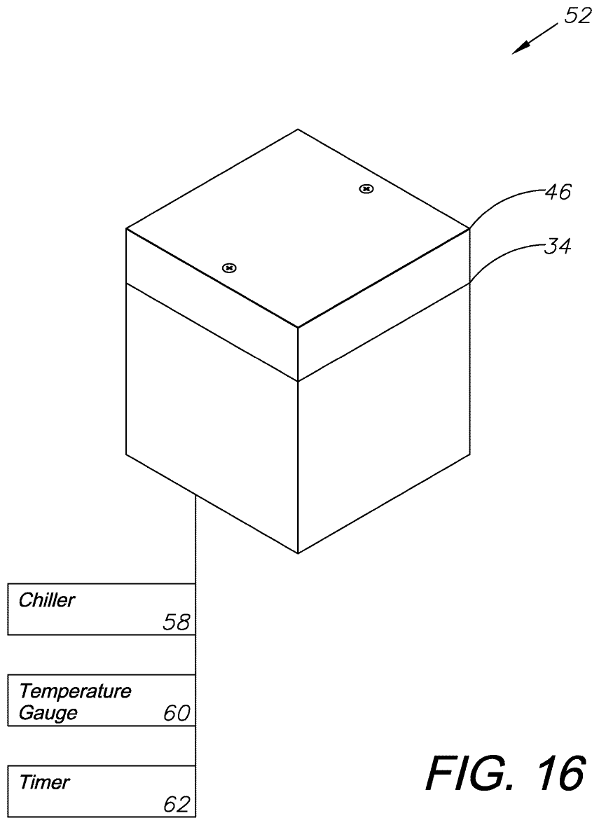

FIG. 15 shows a heating controller 50 with a temperature gauge 54 and timer 56. The mold is heated to an appropriate level to thermoset the coil stent 4. This process can take approximately 15-19 minutes to reach the proper temperature, at which point the mold 34 is held at that temperature for 15 minutes. FIG. 16 shows a chiller 58 with a temperature gauge 60 and timer 62. The chiller 58 sets a temperature of 5.00 degrees Celsius and cools the heated coil stent 4 down to thermoset its shape. A temperature alarm may be included to properly track chilling. Once the temperature gauge 60 indicates a temperature less than 80 degrees Fahrenheit, typically after 15-19 minutes, the chiller 58 is turned off.

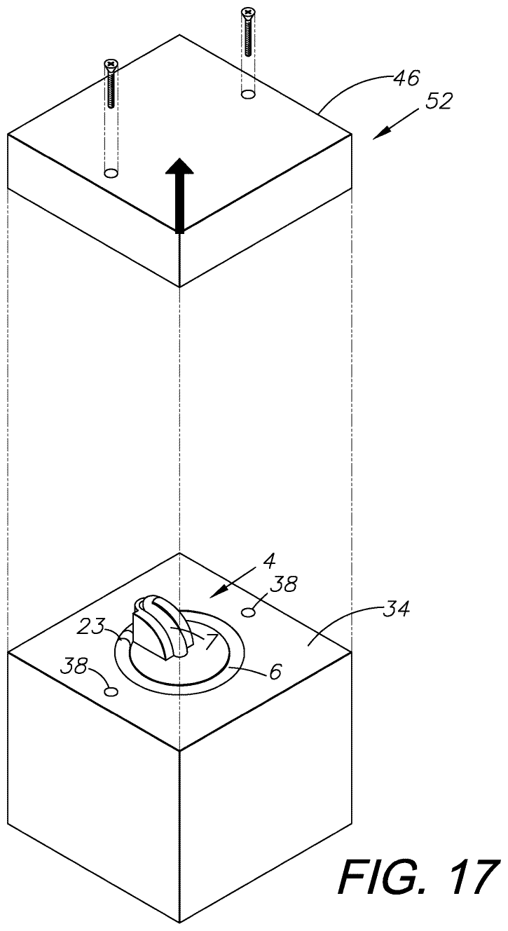

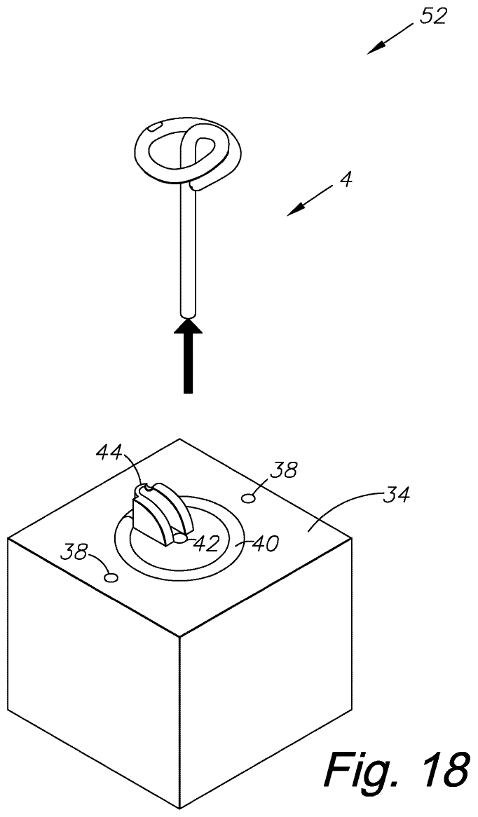

The mold cap 46 is removed as shown in FIG. 17 and the thermoset coil stent 4 is removed from the mold base 34 as shown in FIG. 18. After this step, the coil stent 4 can be placed on a rack for further cooling and should be covered to reduce contamination risks.

### IV. Method 102 of Using Stent System 2

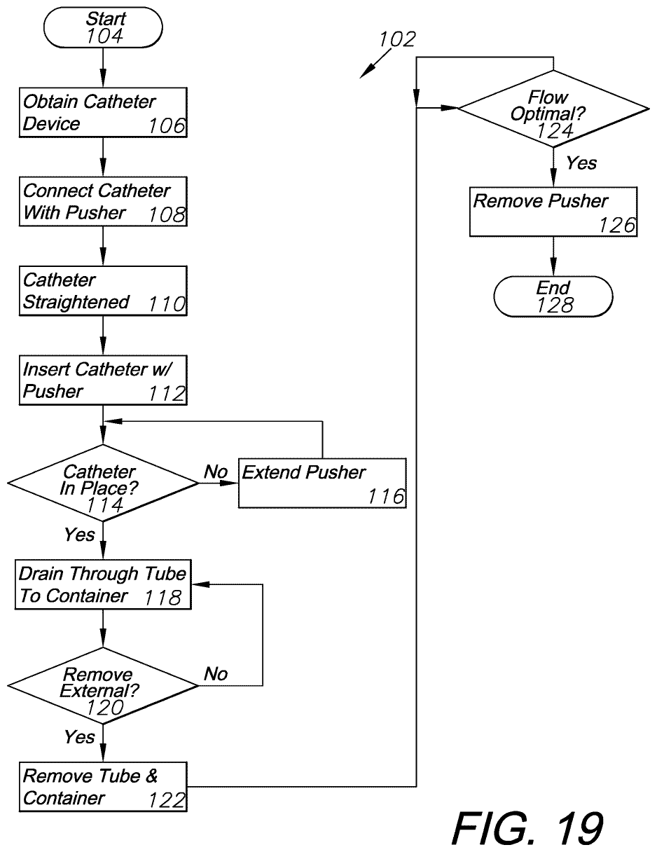

FIG. 19 shows the steps taken in practicing a method 102 of using the coil stent 4 system 2 as described above. The process starts at 104, where the stent 4 is obtained at 106. A pusher 36 is obtained and used with the stent at 108, and the stent 4 is straightened as shown in FIG. 8 at 110. This allows the stent 4 to be inserted into the body at 112 using the pusher 36. In an embodiment, the pusher 36 and the stylet 30 may be connected by a standard luer lock. A check of whether the stent is in place at 114 may require the pusher 36 to be extended at 116 to ensure proper placement of the stent. Once in place at 114, the bladder will drain through the stent 4 eyelet 22 into the stent 4, out through the pusher 36, through a connected tube 27 and into an external container 29 at 118. A determination is made at 120 whether to remove the tube and external container. If not, they remain in place. If so, then the tube and container are removed at 122.

A check is then made at 124 and a determination made whether flow is optimal with the pusher 36 in place. If not, the pusher will remain. If so, the pusher can be removed at 126 so that flow is entirely facilitated using the stent 4 in the body. The process then ends at 128 until such a time that the stent is to be removed.

### Method 152 of Manufacturing System 52 for Manufacture of Stent System 2

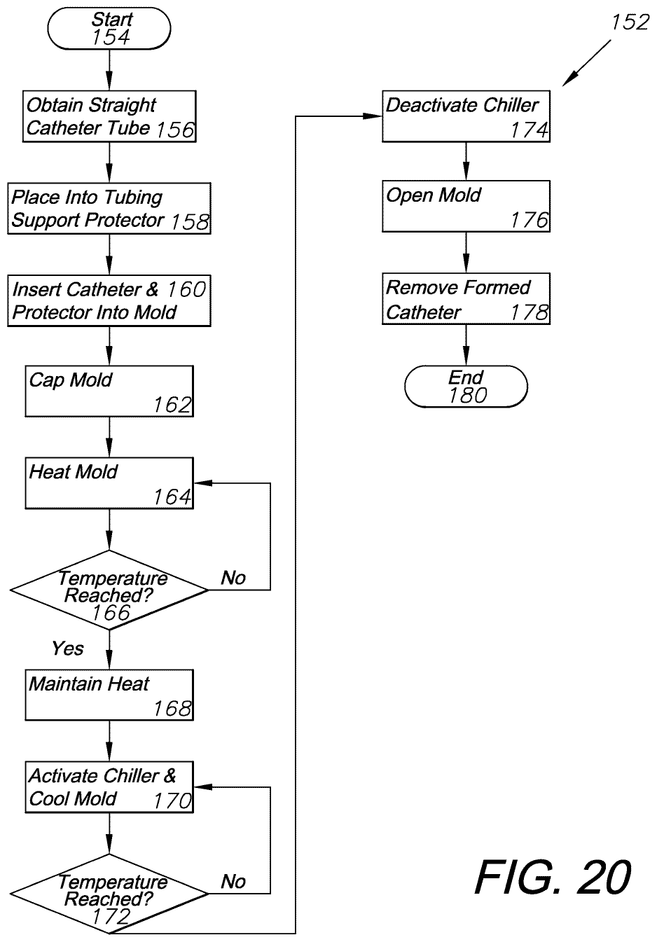

FIG. 20 shows the steps taken in practicing a method 152 of manufacturing the coil stent 4 as described above using the manufacturing system 52. The process starts at 154, where a straight stent tube is obtained at 156. This stent tube should be cut to size, approximately 8.5″ long, and may be outfitted with a tubing support to reduce the risk of tubing kinks during the forming process at 158.

The stent and protector are then inserted into the mold at 160, using the swan neck form 44 and the halo form 40 of the mold base 34. The mold is capped at 162 and heated at 164 as described above. A check using the temperature gauge 54 is made at 166 to determine if the proper temperature has been reached. If not, heating continues. If so, temperature is maintained at 168 for 15 minutes, after which the chiller is activated at 170 to cool the mold.

A check to determine if the mold has reached its cooled temperature below 80 degrees Fahrenheit at 172. If not, chilling continues. If so, then the chiller is deactivated at 174, the mold is opened at 176, and the formed stent 4 is removed at 178, ending the process at 180.

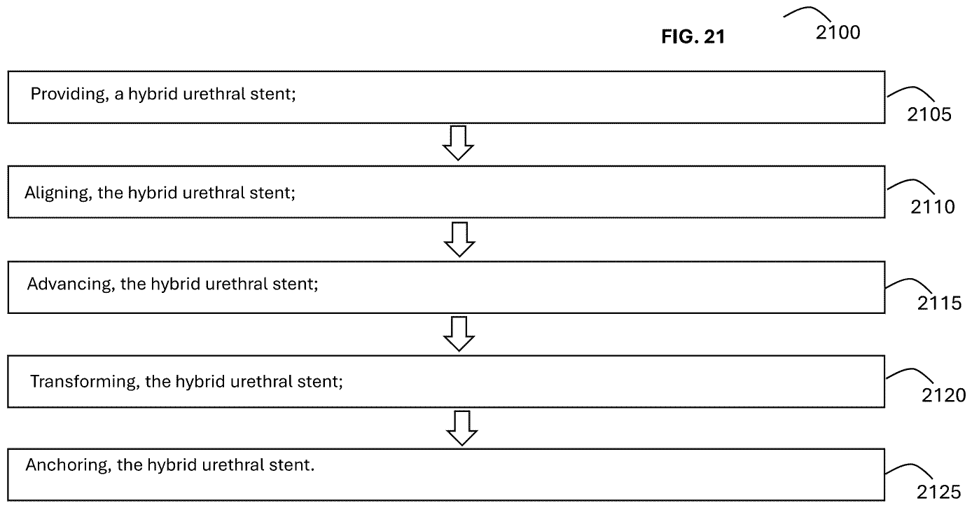

Referring now to FIG. 21, an exemplary method 2100 of inserting a halo-style stent capable of transforming from a first, straight orientation to a second, coiled orientation is illustrated. At step 2105, a hybrid urethral stent system is provided. At step 2110, the hybrid urethral stent system is aligned in its first, straight orientation to facilitate insertion through a urethra. At step 2115, the hybrid urethral stent system is advanced into the urethra such that the proximal end of the stent body is positioned at the bladder base and the distal end of the stent body stents the male prostatic or female urethra and is placed above the external sphincter without penetrating the sphincter. At step 2120, the halo-style stent is transformed into its second, coiled orientation, wherein the single coil assumes a perpendicular configuration relative to the straight stem and the stem. At step 2125, the hybrid urethral stent system is anchored utilizing the memory force of the stabilizing elbow to maintain the hybrid urethral stent system in position within the body.

The stent may be manufactured of Carbothane or other materials which provide long safety use and biocompatibility.

It is to be understood that while certain embodiments and/or aspects of the invention have been shown and described, the invention is not limited thereto and encompasses various other embodiments and aspects.

1. A hybrid urethral stent system capable of transforming from a first, straight orientation to a second, coiled orientation, the stent comprising:
a stent having a stent body including a proximal end and a distal end;
the proximal end of the stent body comprising a single coil having a halo portion terminating into a swan neck element and a right-angle bend, wherein the swan neck element and the right-angle element are configured to form a stabilizing elbow, and wherein the proximal end terminates into a tapered tip;
the distal end of the stent body comprising an extraction thread;
the stabilizing elbow configured to allow for dynamic movement and further configured to provide a memory force to allow for snap back in proximity with a sphincter; the stabilizing elbow terminating into a straight stem culminating in the distal end of the stent body, the straight stem configured to stent a male prostatic or female urethra and to be placed above the external sphincter without penetrating the sphincter during use; and wherein the single coil being perpendicular to the straight portion; a single first eyelet located along a horizontal plane of the single coil; a single second eyelet located along the stabilizing elbow; wherein the stent body functions as a prosthetic or female urethral stent while inserted in a body with the distal end located above the sphincter.

2. The hybrid urethral stent system of claim 1, configured with a non-seating stent body and a plurality of eyelets to facilitate fluid flow both through and around the hybrid urethral stent.

3. The hybrid urethral stent system of claim 1, wherein the swan neck and straight stem configured to stent the male prostatic or female urethra is adapted to permit dual pathway intra-tubular and peri-tubular urine flow.

4. The hybrid urethral stent system of claim 1 further comprising at least an eyelet contained within the single coil and an optional eyelet in the upper portion of the straight stem.

5. The hybrid urethral stent system of claim 3, wherein the peri-tubular urine flow is configured to displace pathogenic microbes from the urinary tract.

6. The hybrid urethral stent system of claim 3, wherein the dual pathway urine flow is configured to produce a higher urine flow rate to flush microbial film and contaminants from the urinary tract.

7. The hybrid urethral stent system of claim 3, wherein the dual pathway urine flow, along with retaining detrusor muscle function, and external and internal sphincter function is configured to reduce post void residual urine in the bladder.

8. The hybrid urethral stent system of claim 3, wherein the system is configured internally to protect the urinary tract mucosal barrier and eliminate the need for an external collection component.

9. The hybrid urethral stent system of claim 1, wherein the second coiled orientation enabling drainage at the bladder base facilitates a complete bladder evacuation.

10. The hybrid urethral stent system of claim 1, wherein placement of the straight stem proximal to the external sphincter is configured to preserve volitional voiding.

11. The hybrid urethral stent system of claim 10, wherein the preservation of volitional voiding further comprises engaging a detrusor muscle contraction functionality and an internal and external sphincter functionality.

12. The hybrid urethral stent system of claim 1, wherein the swan neck flexing feature allows the distal stem to traverse the external sphincter to promote bladder emptying in neurogenic and female patients in a state of urine retention.

13. The hybrid urethral stent system of claim 1, wherein the system is configured to accommodate a urethral length from approximately 1 centimeter to 30 centimeters.

14. The hybrid urethral stent system of claim 1, wherein the stent body is comprised of a material selected from the group consisting of: carbothane, silicone, coated latex, hydrogel, polyurethane, polyvinyl chloride (PVC), nylon, biocompatible material and an antimicrobial coating.

15. The hybrid urethral stent system of claim 1, wherein the hybrid urethral stent is configured to deliver a pharmaceutical medication.

16. A method of inserting a hybrid urethral stent capable of transforming from a first, straight orientation to a second, coiled orientation, the method comprising:
providing the hybrid urethral stent having a stent body including a proximal end and a distal end; the proximal end of the stent body comprising a single coil terminating into a swan neck element forming a right-angle bend, wherein the right-angle element is configured to form a stabilizing elbow, and wherein the proximal end terminates into a tapered tip; the distal end of the stent body comprising straight stem and an extraction thread; the stabilizing elbow configured to allow for dynamic movement and further configured to provide a memory force to allow for snap back in proximity with a sphincter; the stabilizing elbow terminating into a straight stem culminating in the distal end of the stent body, the straight stem configured to stent a male prostatic or female urethra and to be placed above the external sphincter at a base of a bladder without penetrating the sphincter during use; and wherein the single coil being perpendicular to the straight portion; a single first eyelet located along a horizontal plane of the single coil; a single second eyelet located along the stabilizing elbow; and wherein the stent body functions as a hybrid prosthetic or female urethral stent while inserted in a body with the distal end located above the sphincter; aligning, the hybrid urethral stent system stent in its first, straight orientation to facilitate insertion through a urethra; advancing, the hybrid urethral stent system into the urethra such that the proximal end of the stent body is positioned at the bladder base and the distal end of the stent body stents the urethra and is placed above the external sphincter without penetrating the sphincter; transforming, the hybrid urethral stent system in its second, coiled orientation, wherein the single coil assumes a perpendicular configuration relative to the straight stem and the stabilizing elbow conforms to the anatomy near the bladder neck; and anchoring, the hybrid urethral stent system utilizing the memory force of the stabilizing elbow to maintain the hybrid urethral stent system in position within the body.

17. The method of claim 16, further comprising advancing the hybrid urethral stent system into the urethra utilizing a pusher including a stylet containing a guidewire, wherein the guidewire is configured to guide the hybrid urethral stent system into place within the bladder base.

18. The method of claim 17, further comprising removing the stylet containing the guidewire thereby transforming the hybrid urethral stent system into its second, coiled orientation.

19. The method of claim 16, further comprising maintaining the halo-style stent in the anchored position within the body for a duration of up to 30 days.

20. The method of claim 16 further comprising transforming, the hybrid urethral stent system in its first, straight orientation to its second, coiled orientation to initiate urine flow.
